(19) United States
(12) Patent Application Publication
(10) Pub. No.: US 2025/0272607 A1
(43) Pub. Date: Aug. 28, 2025

(54) UTILIZING A TRACTABILITY MACHINE LEARNING MODEL TO GENERATE TRACTABILITY SCORES FOR A MULTI-DOMAIN MACHINE LEARNING MODEL FOR IMPROVED MACHINE LEARNING PREDICTIONS

(71) Applicant: Recursion Pharmaceuticals, Inc., Salt Lake City, UT (US)

(72) Inventors: Seyed Ali Madani Tonekaboni, Toronto (CA); Stephen Scott MacKinnon, Burlington (CA); David Kuter, Ottawa (CA); Nil Sahin, Toronto (CA)

(21) Appl. No.: 18/590,057

(22) Filed: Feb. 28, 2024

(51) Int. Cl. G06N 20/00; G16B 15/20; G16B 15/30; G16B 40/20

(52) U.S. Cl. CPC G06N 20/00; G16B 15/20; G16B 15/30; G16B 40/20

(57) ABSTRACT

The present disclosure relates to systems, non-transitory computer-readable media, and methods that utilize a multi-domain tractability machine learning model to generate tractability scores for a multi-domain machine learning model and further generate improved bioactivity predictions. Indeed, in one or more implementations, the disclosed systems generate a predicted match score between a target protein and a target compound using a compound-protein interaction machine learning model. For instance, the disclosed systems generate a protein-model tractability score that indicates a measure of accuracy of the compound-protein interaction machine learning model relative to the target protein. Moreover, in some instances, the disclosed systems utilize the protein-model tractability score by providing the protein-model tractability score in conjunction with the predicted match score or the target protein or the disclosed systems generate a bioactivity prediction from the predicted match score and the protein-model tractability score.

### BACKGROUND

Recent years have seen significant developments in hardware and software platforms for training and utilizing machine learning models for generating predictions. For example, conventional systems utilize large volumes of training data to teach machine learning models to generate intelligent predictions corresponding to complex biological interactions between genes, compounds, and/or proteins. Despite these recent advances, conventional systems suffer from a number of technical deficiencies, particularly with regard to accuracy, efficiency, and operational inflexibility in implementing machine learning technologies.

### SUMMARY

Embodiments of the present disclosure provide benefits and/or solve one or more of the foregoing or other problems in the art with systems, non-transitory computer-readable media, and methods for utilizing a tractability machine learning model to generate tractability scores for a multi-domain machine learning model for generating improved predictions. For example, the disclosed systems utilize the tractability machine learning model to generate a tractability score for a multi-domain machine learning model, such as a compound-protein interaction machine learning model (e.g., which generates a predicted match/binding score between a target protein and a target compound). The disclosed systems can utilize the tractability machine learning model to generate a protein-model tractability score that indicates a measure of accuracy of the compound-protein machine learning model in generating match predictions relative to the target protein. The disclosed systems can utilize the protein-model tractability score in a variety of downstream applications. For example, the disclosed systems can provide the protein-model tractability score via user interfaces of client devices in conjunction with a predicted match score or target protein to improve context and interpretation accuracy of the multi-domain machine learning model. Moreover, in some instances, the disclosed systems utilize the protein-model tractability score as a signal for analysis in conjunction with other models to generate a bioactivity prediction (e.g., by filtering or weighting the predicted match score with the protein-model tractability score).

Additional features and advantages of one or more embodiments of the present disclosure are outlined in the description which follows, and in part will be obvious from the description, or may be learned by the practice of such example embodiments.

### BRIEF DESCRIPTION OF THE DRAWINGS

The detailed description provides one or more embodiments with additional specificity and detail through the use of the accompanying drawings, as briefly described below.

FIG. 1 illustrates a multi-domain tractability system generating a protein-model tractability score and utilizing the protein-model tractability score in accordance with one or more embodiments.

FIG. 2 illustrates an example diagram of the multi-domain tractability system generating a bioactivity prediction in accordance with one or more embodiments.

FIGS. 3A-3B illustrates an example graphical user interface of the multi-domain tractability system in accordance with one or more embodiments.

FIG. 4 illustrates an example diagram of the multi-domain tractability system training a protein tractability machine learning model in accordance with one or more embodiments.

FIG. 5 illustrates an example environment of the multi-domain tractability system in accordance with one or more embodiments.

FIG. 6 illustrates an example series of acts for transmitting a compound discovery status update notification in accordance with one or more embodiments.

FIG. 7 illustrates a block diagram of a computing device for implementing one or more embodiments.

### DETAILED DESCRIPTION

This disclosure describes one or more embodiments of a multi-domain tractability system 102 that utilizes a tractability machine learning model to generate tractability scores for a multi-domain machine learning model for generating improved machine learning predictions. For example, in one or more implementations the multi-domain tractability system 102 utilizes a protein tractability machine learning model to predict the accuracy of a compound-protein interaction machine learning model for a target protein. To illustrate, the multi-domain tractability system 102 trains the protein tractability machine learning model utilizing ground truth binding data relative to binding predictions generated by the compound-protein interaction machine learning model. The multi-domain tractability system 102 can utilize the trained protein tractability machine learning model to analyze an additional protein (e.g., an unseen protein during training) and corresponding protein features to predict the confidence or tractability of the compound-protein interaction machine learning model for the additional protein. The multi-domain tractability system 102 can use a protein-model tractability score (e.g., that indicates the accuracy or confidence of a model for a specific target) in a variety of downstream applications. For instance, the multi-domain tractability system 102 utilizes the protein-tractability score as a visual element within a client device interface to give context to additional model predictions, as a filter or weighting schema for utilizing a compound-protein interaction prediction, and/or as input to additional models.

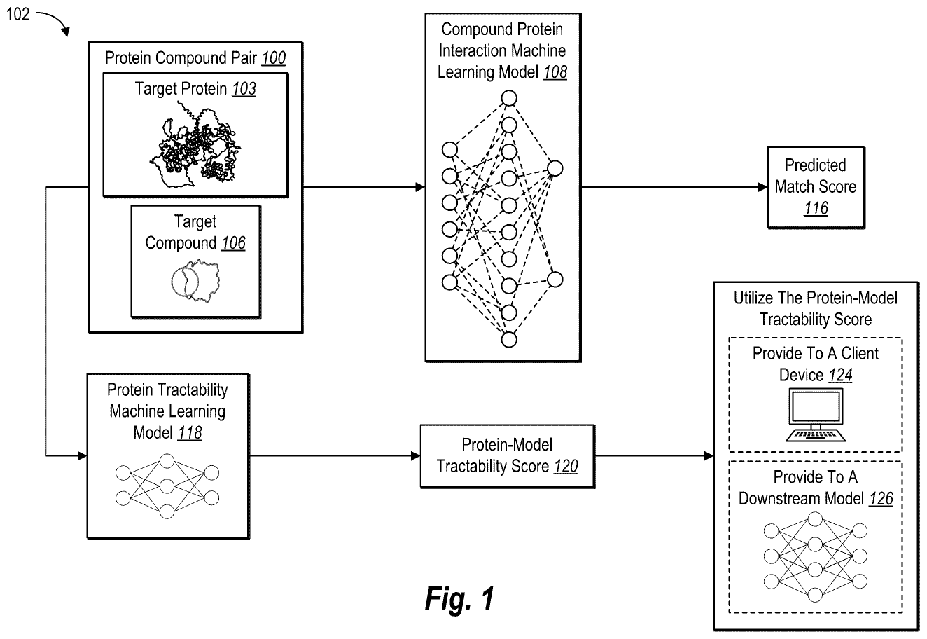

As mentioned above, in one or more embodiments, the multi-domain tractability system predicts the confidence or tractability of binding predictions in the compound-protein space utilizing a compound-protein interaction machine learning model. As shown in FIG. 1, a multi-domain tractability system 102 utilizes a compound-protein interaction machine learning model 108 to generate a predicted match score 116 and further utilizes a protein tractability machine learning model 118 to generate a protein-model tractability score 120. The multi-domain tractability system 102 can then utilize the protein-model tractability score 120 in a variety of downstream models.

As illustrated in FIG. 1, the multi-domain tractability system 102 utilizes the compound-protein interaction machine learning model 108 to generate the predicted match score 116 from a protein-compound pair 100. Specifically, the protein-compound pair 100 includes a target protein 103 and a target compound 106. As used herein, the term “target protein” refers to a protein of interest or focus with regard to an experiment, assay, process, protocol, or analysis (e.g., a protein structure within a cell investigated for treatment of a disease or associated with a target bioactivity). For example, a target protein can include enzymes, receptors, transporters, or other proteins involved in cellular function. For instance, the target protein 103 can include multiple binding sites (e.g., locations or pockets for potentially binding to compounds).

In one or more embodiments, the target compound 106 includes an experimental molecule (e.g., a molecule of interest for a particular treatment or bioactivity). For instance, in some embodiments the target compound 106 has the potential to interact with a biological substrate such as a protein or gene that is associated with the particular disease or condition. In some embodiments the multi-domain tractability system 102 identifies the target compound 106 from machine learning representations of the target protein 103. Furthermore, in some embodiments the multi-domain tractability system 102 identifies the target compound 106 from identifying the target protein 103. For instance, from identifying a protein of interest that has a high correlation with a particular disease or condition, the multi-domain tractability system 102 further identifies the target compound 106 with a statistically significant relationship with the target protein 103.

In one or more embodiments, the multi-domain tractability system 102 utilizes a machine learning model to generate vector representations of the protein-compound pair 100. As used herein, “a machine learning model” includes a computer algorithm or a collection of computer algorithms that can be trained and/or tuned based on inputs to approximate unknown functions. For example, a machine learning model can include a computer algorithm with branches, weights, or parameters that changed based on training data to improve for a particular task. Thus, a machine learning model can utilize one or more learning techniques to improve in accuracy and/or effectiveness. Example machine learning models include various types of decision trees (e.g., Lightgbm, XGBoost, and Random Forest), support vector machines, Bayesian networks, random forest models, or neural networks (e.g., deep neural networks).

Similarly, a neural network includes a machine learning model of interconnected artificial neurons (e.g., organized in layers) that communicate and learn to approximate complex functions and generate outputs based on a plurality of inputs provided to the model. In some instances, a neural network includes an algorithm (or set of algorithms) that implements deep learning techniques that utilize a set of algorithms to model high-level abstractions in data. To illustrate, in some embodiments, a neural network includes a convolutional neural network, a recurrent neural network (e.g., a long short-term memory neural network), a transformer neural network, a generative adversarial neural network, a graph neural network, a diffusion neural network, a fully-connected neural network, or a multi-layer perceptron. In some embodiments, a neural network includes a combination of neural networks or neural network components.

As used herein a “compound-protein interaction machine learning model” refers to a machine learning model that analyzes compounds and protein to generate a prediction. For instance, the compound-protein interaction machine learning model 108 includes a machine learning model that generates predictions regarding binding probabilities for a compound and a protein. Thus, the compound-protein interaction machine learning model 108 includes a deep neural network trained to generate binary predictions (e.g., classifications) and/or match scores between a molecule and protein pocket binding site.

In some embodiments, the multi-domain tractability system 102 utilizes the compound-protein interaction machine learning model 108 to generate vector representations (e.g., which can include a first vector representation of proteins and a second vector representation of compounds). As used herein, the term “vector representation” refers to a mathematical representation of features (e.g., for protein and compound features). Specifically, the vector representations include a conversion to represent protein and compound features in a numerical feature space for further machine learning analysis.

In one or more embodiments, as part of generating the vector representations, the multi-domain tractability system 102 utilizes compound features corresponding to a compound. For example, compound features include properties of a compound such as chemical structure (e.g., reactivity), molecular weight, lipophilicity, aromaticity, target binding affinity (ability to bind to an intended target), and selectivity. In one or more embodiments, as part of generating the vector representations, the multi-domain tractability system 102 utilizes protein features. For example, protein features include properties of a protein such as protein structure (e.g., primary, secondary, tertiary), active site, binding sites, protein-ligand interactions, hydrophobicity, and stability.

To illustrate, the multi-domain tractability system 102 generates the first vector representation to represent the target protein 103 and the second vector representation to represent the target compound 106. Specifically, the multi-domain tractability system 102 learns compatibilities between the first vector representation and the second vector representation to generate the predicted match score 116. As used herein, the term “predicted match score” refers to a metric, score, or probability corresponding to a compound and a protein (generated by a machine learning model). For example, the predicted match score 116 can include a metric indicating a probability that a compound will bind to a protein or subpart of a protein (e.g., protein domain or particular binding site referred to herein as a protein-pocket).

As further shown, the multi-domain tractability system 102 utilizes the protein tractability machine learning model 118 to generate the protein-model tractability score 120 from the target protein 103. As used herein, the term “tractability machine learning model” refers to a machine learning model trained to generate a confidence or tractability of another model. Specifically, the multi-domain tractability system 102 utilizes the tractability machine learning model to predict the accuracy of another machine learning model for a particular target/domain. For example, the multi-domain tractability system 102 utilizes the tractability machine learning model to generate predictions as classifications and/or confidence scores. For instance, the multi-domain tractability system 102 utilizes the tractability machine learning model to generate predictions and utilizes the predictions in downstream applications such as providing the prediction to a user interface or as input to additional models. In other words, the tractability machine learning model generates predictions of accuracy for multi-domain models in relation to a specific target (e.g., a single domain of the multi-domain model).

Further, as used herein, the term “protein tractability machine learning model” refers to a machine learning model trained to generate a confidence or tractability of a model in generating predictions in the protein space. Specifically, the multi-domain tractability system 102 utilizes the protein tractability machine learning model 118 to predict the accuracy of a model in relation to a target protein. For example, the multi-domain tractability system 102 utilizes the protein tractability machine learning model 118 to generate a confidence or tractability for the compound-protein interaction machine learning model 108 with regard to a particular protein (e.g., an individual protein, a protein binding site, and/or a protein class/group).

As used herein, the term “protein-model tractability score” refers to a score that indicates a measure of accuracy, confidence, or performance of a protein model relative to a target protein. Specifically, the protein-model tractability score 120 includes a score that indicates high confidence or low confidence (e.g., ranging from 0-1) or includes a binary score that indicates sufficiency or insufficiency. For example, the protein-model tractability score can include a 0.65 score for a target protein (e.g., EGFR-Epidermal Growth Factor Receptor 1), which means there is a 0.65 confidence for a specific protein model in relation to EGFR.

The multi-domain tractability system 102 can utilize a variety of protein-model tractability scores (e.g., percent confidence or a normalized accuracy score). For instance, in some implementations, the multi-domain tractability system 102 generates the protein-model tractability score 120 as a ROC-AUC measure (e.g., Receiver Operating Characteristic-Area Under the Curve, which indicates the trade-off between true positive rates and false positive rates, a higher AUC indicates better performance for the model relative to the target protein; for example, the multi-domain tractability system 102 generates AUC scores for the protein features of the target protein 103), a Mann-Whitney U Test (e.g., indicates difference of model predictions for positive and negative instances of the target protein), F1 score (e.g., the mean of precision and recall), a confusion matrix (e.g., which indicates a breakdown of true positives, true negatives, false positives, and false negatives), or an Area Under the Precision-Recall Curve (e.g., similar to ROC-AUC, assesses the trade-off between precision and recall at different thresholds).

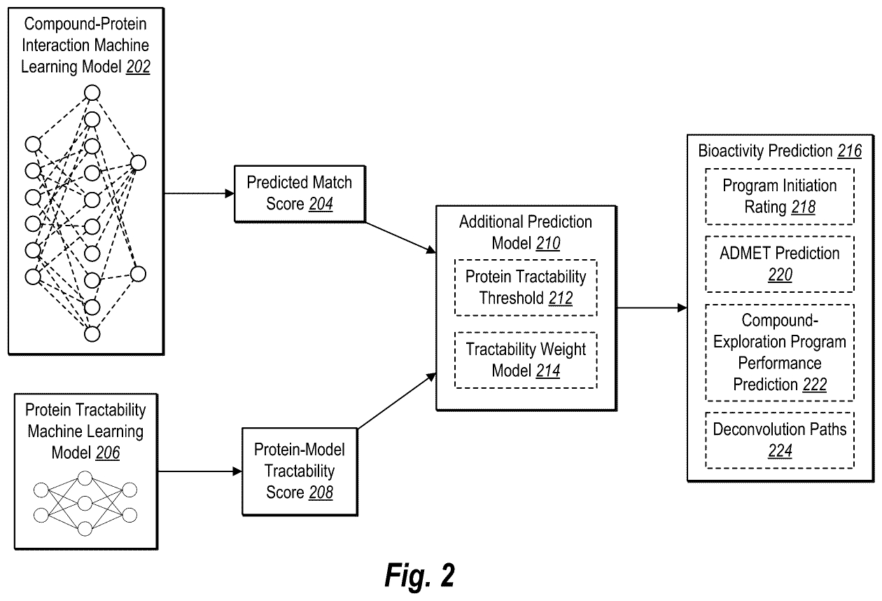

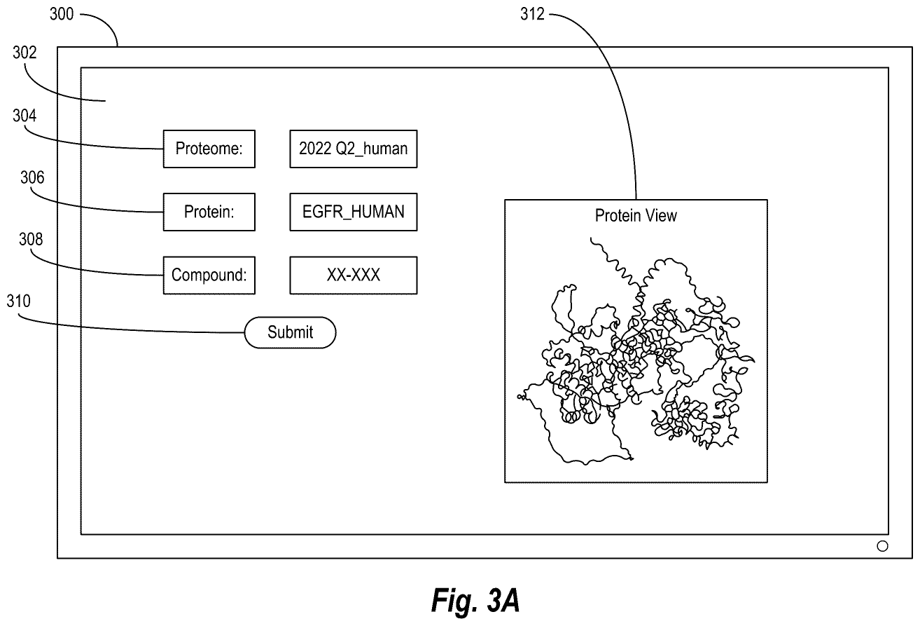

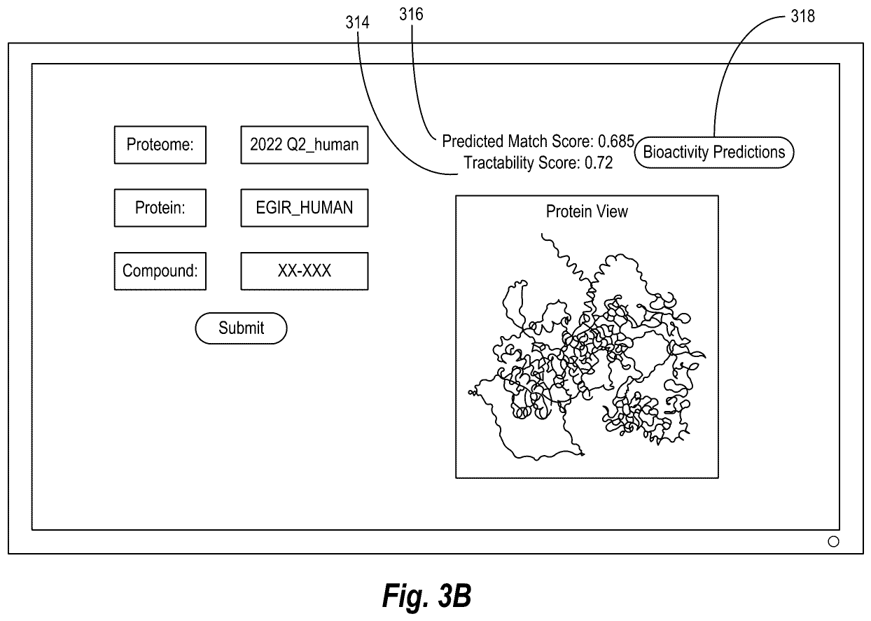

As shown in FIG. 1, the multi-domain tractability system 102 receives the pocket features and the protein features of the target protein 103 and utilizes the protein tractability machine learning model 118 to generate the protein-model tractability score 120. As further illustrated in FIG. 1, the multi-domain tractability system 102 utilizes the protein-model tractability score 120. Specifically, in some embodiments, the multi-domain tractability system 102 provides the protein-model tractability score 120 to a client device 124. In some embodiments, the multi-domain tractability system 102 provides the protein-model tractability score 120 to a downstream model 126. For example, the multi-domain tractability system 102 utilizes the downstream model 126 to generate bioactivity predictions. Additional details of the multi-domain tractability system 102 providing the protein-model tractability score 120 is given below in FIGS. 3A-3B and details of the bioactivity predictions are given below in the description of FIG. 2.

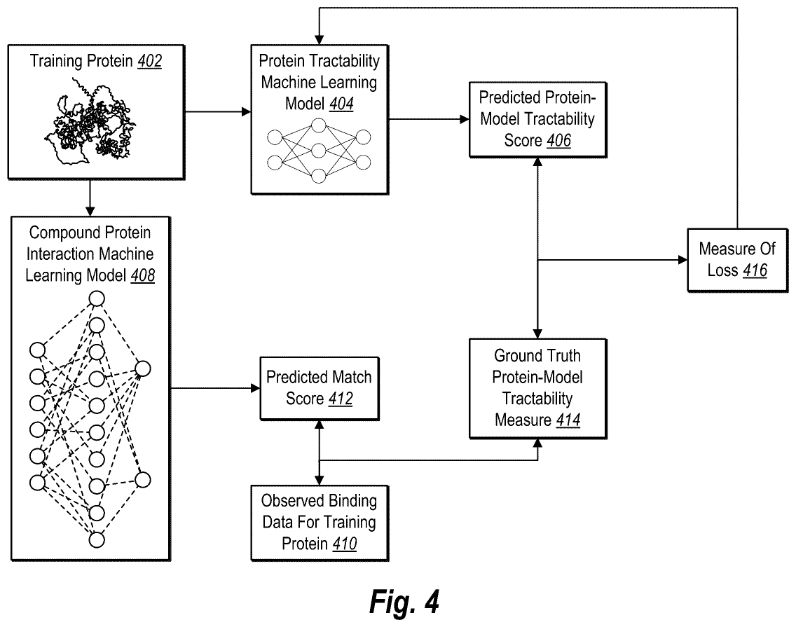

In some embodiments, the multi-domain tractability system 102 generates the protein-model tractability score 120 with respect to a certain species (e.g., human, animal, agricultural, and other non-human organisms). Specifically, the multi-domain tractability system 102 can generate a protein-model tractability score for a protein of a particular specific species (e.g., the score indicating the accuracy of a specific model in the protein domain for a specific species). Specific details of training the protein tractability machine learning model 118 for generating protein-model tractability scores is given below in the description of FIG. 4.

Moreover, in some embodiments, the multi-domain tractability system 102 generates a tractability score for a specific model (e.g., the compound interaction machine learning model 108) across an entire species. For instance, the multi-domain tractability system 102 utilizes the protein tractability machine learning model 118 to generate protein-model tractability scores for an entire proteome specific to a species. In doing so, the multi-domain tractability system 102 combines the protein-model tractability scores for the entire proteome and generates a species tractability score (e.g., 0.85 score of the compound protein interaction machine learning model in the protein domain for a cow).

As described above, FIG. 1 illustrates the multi-domain tractability system 102 generating a tractability score specific to the protein space. As alluded to above, in one or more embodiments, the multi-domain tractability system 102 generates tractability scores for additional domain spaces. Specifically, the multi-domain tractability system 102 generates tractability scores for the compound space. For example, the multi-domain tractability system 102 identifies chemotypes (e.g., clusters of compounds in the compound space that share similar chemical substructures), and the multi-domain tractability system 102 further generates a tractability score that indicates a level of confidence or tractability of the chemotype.

Moreover, although the above description relates to the protein space, in some embodiments, the multi-domain tractability system 102 generates tractability scores for the chemophenomics space and the phenomic space. Specifically, the multi-domain tractability system 102 generates a tractability score in the chemophenomics space for a gene (in a chemical-genetic space), a compound (in the chemical-genetic space), a protein (in a genetic-protein space), or a gene (in the genetic-protein space), etc. In other words, the multi-domain tractability system 102 can generate a tractability score for a variety of multimodal models in relation to a single mode or domain of the multimodal models.

For instance, the multi-domain tractability system 102 performs cell perturbations and captures phenomic digital images of the perturbed cells. Specifically, the multi-domain tractability system 102 performs a machine learning analysis on the digital images portraying perturbed cells to generate embeddings from the phenomic digital images and compares the embeddings to identify inter-relationships between genes, proteins, compounds, and/or diseases. Thus, the multi-domain tractability system 102 generates a tractability score for a single domain (e.g., a gene or a compound) of a machine learning model that generates phenomic embeddings. To illustrate, the multi-domain tractability system 102 generates phenomic embeddings as described in application Ser. No. 18/392,989 UTILIZING MACHINE LEARNING AND DIGITAL EMBEDDING PROCESSES TO GENERATE DIGITAL MAPS OF BIOLOGY AND USER INTERFACES FOR EVALUATING MAP EFFICACY filed on Dec. 21, 2023, which is fully incorporated by reference herein.

As mentioned briefly above, conventional systems suffer from a number of technical deficiencies with regard to implementing computing devices. For example, conventional systems often generate inaccurate machine learning predictions. Indeed, although conventional systems can utilize machine learning models to generate some biological predictions, such predictions are often inaccurate because conventional systems often fail to have sufficient binding information for a wide range of proteins. Thus, conventional systems often generate multi-domain predictions for a target protein (e.g., unseen proteins during training) that can be based on insufficient data, leading to the generation of inaccurate predictions.

Furthermore, conventional systems are often inefficient. For example, conventional systems often determine to initiate bioactivity programs for one or more target proteins based on upstream predictions. Indeed, as mentioned above, upstream predictions in conventional systems are often inaccurate due to being based on insufficient data. As such, conventional systems initiate bioactivity programs based on faulty predictions, which consumes and wastes a significant number of computational resources.

Moreover, conventional systems often require excessive user interactions, processes, and user interfaces to analyze the efficacy or accuracy of machine learning models. To illustrate, conventional systems can utilize implementing computing devices to employ a testing protocol for comparing predictions to measured results. Such systems can then generate testing results and provide such results for display via a series of user interfaces for different models and different experimental targets. Such systems require excessive time, computing resources (e.g., processing power and memory) to establish and implement such processes as well as provide and navigate through user interfaces to identify and act on pertinent information.

Conventional systems are also operationally inflexible. Indeed, conventional systems often rigidly utilize machine learning predictions without sufficient context. For example, conventional systems may be able to predict a potential relationship between a protein and compound, however, conventional systems are often rigidly focused on the resulting prediction (e.g., a classification or score) without contextual information regarding the machine learning model and efficacy of the prediction. Thus, conventional systems suffer from varying degrees of uncertainty with regards to machine learning predictions which leads to rigid utilization and analysis for downstream tasks.

As suggested by the foregoing discussion, the multi-domain tractability system 102 provides a variety of technical advantages relative to conventional systems. For example, the multi-domain tractability system 102 can improve accuracy of implementing computing devices including utilization of bioactivity predictions for downstream models or tasks. As mentioned above, conventional systems suffer from generating multi-domain predictions for a target protein based on insufficient data. In contrast, the multi-domain tractability system 102 can generate a protein-model tractability score (e.g., using a protein tractability machine learning model) that indicates a measure of accuracy of a compound-protein interaction machine learning model relative to a target protein. This allows the multi-domain tractability system 102 to analyze and consider relative tractability or confidence of machine learning predictions in downstream models or tasks to improve accuracy of implementing devices in generating bioactivity predictions.

To illustrate, in some embodiments, the multi-domain tractability system 102 provides the protein-model tractability score for display via client devices in conjunction with a predicted match score or a target protein to allow client devices to consider and incorporate the protein-model tractability score in utilizing a particular prediction. Moreover, in some embodiments, the multi-domain tractability system 102 improves accuracy of downstream models by utilizing the protein-model tractability score as an additional signal in generating bioactivity predictions. Thus, for example, the multi-domain tractability system 102 utilizes the protein-model tractability score as part of a filter or weighting model to account for the accuracy of the predicted match score for a target protein in generating bioactivity predictions.

In addition to the accuracy improvements, in some embodiments, the multi-domain tractability system 102 improves the efficiency of conventional systems. As mentioned, conventional systems often require excessive user interactions, processes, and user interfaces to analyze the efficacy or accuracy of machine learning models. In contrast, the multi-domain tractability system 102 can utilize a protein tractability machine learning model to generate protein-model tractability scores and provide the protein-model tractability scores to efficiently provide pertinent context for binding predictions or match scores from a compound protein interaction machine learning model. Indeed, the multi-domain tractability system 102 can avoid the time and computer resources needed to organize and implement complex testing protocols by utilizing a tractability machine learning model to generate a prediction indicating the accuracy of the compound protein interaction machine learning model with regard to a particular protein. Moreover, the multi-domain tractability system 102 can avoid the time and computing resources associated with excessive user interfaces and user interactions by providing the protein model tractability score in conjunction with predictions resulting from the compound protein interaction machine learning model. Furthermore, the multi-domain tractability system 102 can generate bioactivity predictions and initiate compound exploration processes based on more complete signals indicating the accuracy of binding predictions/match scores. This allows the multi-domain tractability system 102 to avoid wasting additional computational resources.

Relatedly, in some embodiments, the multi-domain tractability system 102 improves upon operational flexibility. For example, the multi-domain tractability system 102 generates the protein-model tractability score to indicate to one or more downstream models or devices the accuracy of a generated prediction in relation to a specific target. Thus, the multi-domain tractability system 102 can include important contextual data surrounding one or more predictions to perform bioactivities more accurately and efficiently. Indeed, rather than rigidly relying on a binding predictions/match scores, the multi-domain tractability system 102 can generate and provide a protein-model tractability score to flexibly apply such predictions based predicted model tractability for a particular protein space.

As just mentioned, the multi-domain tractability system 102 generates a bioactivity prediction utilizing the protein-model tractability score. For example, FIG. 2 shows the multi-domain tractability system 102 utilizing an additional prediction model to generate a bioactivity prediction from the protein-model tractability score in accordance with one or more embodiments.

As shown in FIG. 2, the multi-domain tractability system 102 utilizes a compound-protein interaction machine learning model 202 to generate a predicted match score 204 and utilizes a protein tractability machine learning model 206 to generate a protein-model tractability score 208. For instance, the multi-domain tractability system 102 utilizes the protein tractability machine learning model 206 to process local protein features (e.g., features regarding local protein pockets, such as binding site features, graph descriptions, pocket shapes, atom type descriptors), global protein features (e.g., features regarding a protein as a whole, such as the structures and sequence of a protein), protein functional features (e.g., functions or purposes of a particular protein or protein pocket).

Furthermore, FIG. 2 shows the multi-domain tractability system 102 utilizing an additional prediction model 210. As used herein, the term “additional prediction model” includes or refers to one or more machine learning models utilized to generate one or more predictions.

As shown, the additional prediction model 210 includes in some embodiments, a protein tractability threshold 212. As used herein, the term “protein tractability threshold” refers to a threshold that indicates when a protein has a satisfactory tractability versus when it has an unsatisfactory tractability. Specifically, the multi-domain tractability system 102 determines a predetermined number as the protein tractability threshold (e.g., 0.5). For example, the multi-domain tractability system 102 compares the protein-model tractability score 208 to the protein tractability threshold 212, for scores that satisfy the threshold (e.g., greater than or equal to 0.5), the multi-domain tractability system 102 generates a bioactivity prediction 216 that reflects that indication (e.g., and vice-versa).

In some embodiments, if the protein-model tractability score 208 fails to satisfy the protein tractability threshold 212 (e.g., less than 0.5), the multi-domain tractability system 102 does not perform any downstream analysis or generate additional predictions for the target protein. In other words, the multi-domain tractability system 102 drops the target protein from consideration (e.g., by not generating a subsequent bioactivity prediction) because the confidence or tractability of the target protein fails to reach the 0.5 threshold. For example, in some implementations, the multi-domain tractability system 102 utilizes the protein-model tractability score 208 as a confidence machine learning model as a filter in generating bioactivity predictions, as described in UTILIZING COMPOUND-PROTEIN MACHINE LEARNING REPRESENTATIONS TO GENERATE BIOACTIVITY PREDICTIONS, U.S. application Ser. No. 18/505,728, filed Nov. 9, 2023, which is incorporated by reference in its entirety herein.

As also shown, the additional prediction model 210 includes in some embodiments, a tractability weight model 214. Specifically, the multi-domain tractability system 102 utilizes the tractability weight model 214 to assign a weight to the protein-model tractability score 208. As used herein, the term “weight” refers to a significance or value measure (e.g., based on the protein-model tractability score 208). Specifically, the multi-domain tractability system 102 generates the protein-model tractability score 208 and uses the protein-model tractability score 208 as a weight for generating the bioactivity prediction 216. For example, for a high protein-model tractability score (e.g., greater than 0.90), the multi-domain tractability system 102 weighs a predicted match score (e.g., the predicted match score 204) proportionally. In other words, the multi-domain tractability system 102 favors the predicted match score 204 for a high protein-model tractability score when generating the bioactivity prediction 216.

In one or more embodiments, the multi-domain tractability system 102 utilizes the tractability weight model 214 as a signal for utilization in a machine learning model. Thus, the tractability weight model 214 can include learned parameter weights in a machine learning model for processing the protein-model tractability score 208 (e.g., internal weights of layers of a neural network). Specifically, if the multi-domain tractability system 102 generates the protein-model tractability score 208 of 0.65, the multi-domain tractability system 102 process the protein-model tractability score 208 through learned weights of a machine learning model to generate a machine learning prediction. In other words, the multi-domain tractability system 102 utilizes the tractability weight model 214 to assign different levels of importance to features (e.g., the predicted match score 204) which influences the inferences/predictions of the downstream models.

In one or more embodiments, the multi-domain tractability system 102 utilizes the tractability weight model 214 to generate heuristic weights. Specifically, if the multi-domain tractability system 102 generates the protein-model tractability score of 0.65, the multi-domain tractability system 102 can utilize the tractability weight model 214 to reduce the predicted match score 204 by 65%. For example, if the predicted match score 204 is 0.5, the predicted match score 204 scaled according to the heuristic weight is 0.325.

In some embodiments, the multi-domain tractability system 102 utilizes the protein tractability threshold 212 in conjunction with the tractability weight model 214. Specifically, the multi-domain tractability system 102 determines whether the protein-model tractability score 208 satisfies the protein tractability threshold 212. For example, for the protein-model tractability score 208 that satisfies the protein tractability threshold 212, the multi-domain tractability system 102 bolsters the predicted match score 204 (e.g., bolsters by the specific amount of the protein-model tractability score 208, doubles the score, or bolsters by any predetermined amount).

On the contrary, in some embodiments, for the protein-model tractability score 208 that fails to satisfy the protein tractability threshold 212, the multi-domain tractability system 102 reduces the predicted match score 204 (reduces by the specific amount of the protein-model tractability score 208, cuts the score in half, or reduces by any predetermined amount). Accordingly, the multi-domain tractability system 102 can utilize the tractability weight model 214 and the protein tractability threshold 212 to adjust the predicted match score 204 and utilizes the adjusted score in downstream models to generate the bioactivity prediction 216.

Furthermore, in some embodiments, the multi-domain tractability system 102 utilizes different tiers for the protein tractability threshold 212. Specifically, the multi-domain tractability system 102 can implement a low-tier, a mid-tier, and a high-tier. For example, the multi-domain tractability system 102 establishes the low-tier at 0.2, the mid-tier at 0.5, the high-tier 0.75. Moreover, the multi-domain tractability system 102 assigns a predetermined heuristic/parameter weight to adjust the predicted match score 204 (e.g., or any other score) based on the satisfied tier. To illustrate, for the protein-model tractability score 208 that fails to satisfy the low-tier, the multi-domain tractability system 102 can reduce (e.g., assign a parameter weight) the predicted match score 204 by 75%. In contrast, for the protein-model tractability score 208 that satisfies the high-tier, the multi-domain tractability system 102 can increase (e.g., assign a parameter weight) the predicted match score 204 by 25%.

As used herein, the term “bioactivity prediction” refers to a prediction related to a specific target. Specifically, the bioactivity prediction 216 includes a prediction for a target bioactivity, biological feature, or outcome. Thus, for instance the bioactivity prediction 216 can include an ADMET prediction 220, which refers to a prediction corresponding to absorption (e.g., compound/drug entering the bloodstream), distribution (compound/drug being distributed through the body to tissues and organs, such as solubility or permeability of body barriers), metabolism (chemical transformation of a compound/drug within the body), excretion (elimination of a compound/drug from the body), or toxicity (harmful effects of a compound/drug). Thus, the ADMET prediction 220 stands for Absorption, Distribution, Metabolism, Excretion, and Toxicity. Furthermore, as shown a bioactivity prediction can include a program initiation rating 218 or a compound exploration program performance prediction 222 (which is described in more detail below).

In one or more embodiments, the multi-domain tractability system 102 utilizes a language machine learning model to generate rating metrics and further combines the rating metrics to generate the program initiation rating 218, as described in UTILIZING BIOLOGICAL MACHINE LEARNING REPRESENTATIONS AND A LANGUAGE MACHINELEARNING MODEL FOR INITIATING COMPOUND EXPLORATION PROGRAMS, U.S. application Ser. No. 18/521,910, filed Nov. 28, 2023, which is incorporated by reference herein in its entirety. For instance, the multi-domain tractability system 102 receives multiple rating metrics from multiple digital text prompts and combines the rating metrics to determine the program initiation rating 218. Specifically, the program initiation rating 218 indicates whether to initiate one or more compound exploration programs. Moreover, the multi-domain tractability system 102 utilizes the protein-model tractability score 208 as a factor in generating the program initiation rating 218 by adjusting one or more rating metrics according to the protein-model tractability score (e.g., to influence combining the multiple rating metrics to generate the program initiation rating 218).

In some embodiments a “compound exploration program performance prediction” includes a prediction related to initiating a process of identifying and selecting potential chemical compounds or molecules for development into new or enhanced drugs or agents. In some instances, the compound exploration process involves filtering down tens of thousands of compounds to a couple of compounds to target a protein with a compound most effectively.

For instance, a compound exploration program includes utilizing a compound to target a protein involved in an underlying disease and testing many compounds to identify how the compounds interact with the specific target. Additionally, compound exploration programs involve optimizing identified compounds and analyzing results of the compounds applied to the specific target. In some instances, compound exploration programs also involve pre-clinical testing, clinical trials, candidate selection for further testing, and regulatory approval. Thus, the multi-domain tractability system 102 generates the compound exploration program performance prediction 222 to determine whether to initiate one or more compound exploration programs.

To illustrate, in some embodiments, the compound exploration programs include industrial program generation (IPG) and industrial compound generation (ICG). For instance, an industrial program generation (IPG) includes (i) a hit selection to identify statistically strong connections in a biological map to patient-informed phenotypes, (ii) phenomic confirmation (e.g., promising actives are confirmed by automated similarity and concentration-response analytics), (iii) Trekseq confirmation (e.g., compound and gene relationships are confirmed with transcriptomics in the map background), and (iv) Structure-Activity Relationship (SAR) confidence (e.g., actives that behave as a series are identified, and an automated recommendation for expansion is identified). Moreover, in some embodiments, ICG applies to steps subsequent to IPG. Further, in some embodiments ICG includes rapidly searching and expanding from potential hit series in the chemical space (e.g., identified at the IPG stage) and testing the potential hits with various analytical tests (e.g., SAR screens).

As mentioned, in some embodiments, the multi-domain tractability system 102 generates the protein-model tractability score 208 that indicates a high confidence for a target protein. In such instances, the multi-domain tractability system 102 can generate the bioactivity prediction 216 that indicates IPG for the target protein should be initiated. Further, in response to the bioactivity prediction 216, in some instances, the multi-domain tractability system 102 generates a request to order additional compounds that specifically bind to the target protein.

As also shown in FIG. 2, in some embodiments, the bioactivity prediction 216 includes deconvolution paths 224. Specifically, the multi-domain tractability system 102 utilizes the predicted match score 204 and the protein-model tractability score 208 to generate the deconvolution paths 224 to narrow a mechanism of action for a particular compound. For example, the multi-domain tractability system 102 can identify an active compound that has a particular impact on a cell. The multi-domain tractability system 102 can explore the deconvolution paths 224 to identify proteins that indicate the mechanism of action for this particular impact. In doing so, the multi-domain tractability system 102 can generate predicted match scores across different proteins to identify the most likely candidate proteins that explain the impact. The multi-domain tractability system 102 can utilize protein-model tractability scores to narrow the search and improve the deconvolution paths 224 (e.g., by emphasizing match scores that with higher accuracy and deemphasizing match scores with lower accuracy). In other words, the multi-domain tractability system 102 can explore the deconvolution paths 224 to deconvolute a mechanism of action of a compound.

As mentioned above, in some embodiments, the multi-domain tractability system 102 provides for display a tractability score via a graphical user interface. As shown in FIG. 3A, the multi-domain tractability system 102 provides for display via a graphical user interface 302 of a client device 300 an interface to obtain data regarding a protein 306. Specifically, FIG. 3A shows a proteome 304 for the client device 300 to indicate a proteome selection (e.g., 2022 Q2_human), the protein 306 to indicate a protein selection (EGFR_HUMAN), and a compound 308 to indicate a compound selection (e.g., XX-XXX).

For example, the multi-domain tractability system 102 provides one or more options for a user of the client device 300 to select one or more drop-down fields or to manually enter the proteome 304, the protein 306, and the compound 308. Moreover, FIG. 3A shows the multi-domain tractability system 102 providing a protein view 312 of the protein 306 selected by the user of the client device 300. Additionally, FIG. 3A shows an element 310 that reads “submit” and the element 310 is a selectable element to submit a query regarding the proteome 304, the protein 306, and the compound 308.

For instance, in response to selecting the element 310, the multi-domain tractability system 102 utilizes the compound protein interaction machine learning model to generate a predicted match score 316 for the protein 306 (EGFR_HUMAN) and the compound 308. Furthermore, in response to selecting the element 310, the multi-domain tractability system 102 also utilizes the protein tractability machine learning model to generate a protein-model tractability score 314 for the protein 306 (e.g., EGFR_HUMAN). As described above, the multi-domain tractability system 102 takes features of the protein 306 as input to the protein tractability machine learning model to generate the protein-model tractability score 314.

To illustrate, FIG. 3B shows the predicted match score 316 of 0.685 which indicates a protein-compound interaction of 0.685 and shows the protein-model tractability score 314 as 0.72 which indicates a confidence or tractability of the compound protein interaction machine learning model in generating the predicted match score 316 for the protein 306 (e.g., EGFR_HUMAN). Alternatively, the multi-domain tractability system 102 can display the protein-model tractability score 314 in conjunction the target protein (e.g., EGFR_HUMAN) rather than generating the predicted match score 316.

Additionally, FIG. 3B illustrates an additional element 318 that reads “bioactivity predictions.” Specifically, in response to receiving a selection of the additional element 318, the multi-domain tractability system 102 utilizes an additional prediction model to generate additional predictions. For example, the multi-domain tractability system 102 takes the predicted match score 316 and the tractability score 314 and utilizes the additional prediction model to generate one or more bioactivity predictions. To illustrate, the multi-domain tractability system 102 generates the one or more bioactivity predictions and causes the graphical user interface 302 to display the one or more bioactivity predictions. For instance, the multi-domain tractability system 102 causes the graphical user interface 302 to show a program initiation rating, an ADMET prediction, and/or a compound exploration program performance prediction. Moreover, in some instances, the multi-domain tractability system 102 further causes the graphical user interface 302 to show whether the tractability score 314 satisfies one or more tractability thresholds and/or the weight assigned to the predicted match score 316 based on the tractability score 314.

Furthermore, although not shown in FIGS. 3A and 3B, in one or more embodiments, the multi-domain tractability system 102 utilizes the protein tractability machine learning model to generate an additional protein-model tractability score. Specifically, the additional protein-model tractability score also indicates a measure of accuracy of the compound-protein interaction machine learning model relative to an additional target protein. For example, the multi-domain tractability system 102 generates the additional protein-model tractability score for HER2 (Human Epidermal Growth Factor Receptor 2). Moreover, in some embodiments, the multi-domain tractability system 102 generates additional bioactivity predictions for the additional protein-model tractability score.

Additionally, in some embodiments, the multi-domain tractability system 102 generates the bioactivity prediction from the protein-model tractability score and the additional protein-model tractability score. In other words, for a downstream model prediction involving program initiation rating (or some other prediction), the strength of the rating is tied to both the protein-model tractability score and the additional protein-model tractability score. In such cases, the multi-domain tractability system 102 generates the bioactivity prediction by feeding as input both the protein-model tractability score and the additional protein-model tractability score to a machine learning model.

As mentioned above, the multi-domain tractability system 102 trains the protein tractability machine learning model. For example, FIG. 4 shows the multi-domain tractability system 102 utilizing observed binding data to train the protein tractability machine learning model in accordance with one or more embodiments.

As shown in FIG. 4, the multi-domain tractability system 102 utilizes a protein tractability machine learning model 404 to generate a predicted protein-model tractability score 406 from a training protein 402. As used herein, “a training protein” refers to a protein used for training a machine learning model. Specifically, the multi-domain tractability system 102 utilizes the training protein 402 to train the protein tractability machine learning model 404.

As mentioned above, the term “protein-model tractability score” refers to a score that indicates a measure of accuracy of a protein model relative to a target protein. In addition, a “predicted protein-model tractability score” refers to a tractability score utilized during training of the protein tractability machine learning model 404. Specifically, the multi-domain tractability system 102 generates the predicted protein-model tractability score 406 from a protein (being used for training purposes) and compares the predicted protein-model tractability score to a ground truth. For example, the multi-domain tractability system 102 compares the predicted protein-model tractability score 406 with a ground truth protein-model tractability measure 414.

The multi-domain tractability system 102 can utilize a variety of ground truth protein-model tractability measures. For example, in some embodiments, the multi-domain tractability system 102 generates the protein-model tractability score as a Mann-Whitney U-test or a ROC-AUC score. In one or more embodiments, the multi-domain tractability system 102 utilizes a Mann-Whitney U-test (e.g., Wilcoxon rank-sum test) to generate the predicted protein-model tractability score 406 (e.g., by employing a non-parametric test (does not rely on specific distributional assumptions) to assess whether there is a significant difference between positive and negative instances of the training protein 402).

Moreover, in some embodiments, the multi-domain tractability system 102 utilizes a Receiver Operating Characteristic-Area Under the Curve (ROC-AUC) to evaluate the training protein 402 and generate the predicted protein-model tractability score 406. Specifically, the “ROC” portion of ROC-AUC indicates the true positive rate against a false positive rate at different thresholds. Further, the “AUC” portion indicates a single scalar value that summarizes the performance of the classifier across all possible prediction thresholds. For instance, AUC values range from 0-1 where a higher AUC indicates a better classifier performance (e.g., an AUC of 0.5 can suggest that the prediction performs no better than random chance). In other words, the multi-domain tractability system 102 employs various statistical tests or measures to look at the protein features of a specific protein and further determines the false positives versus the true positives for the specific protein to generate the tractability score.

As used herein, the term “ground truth protein-model tractability measure” refers to a measure verified by real-world data. Specifically, multi-domain tractability system 102 generates the ground truth protein-model tractability measure 414 by generating a predicted match score 412 for a protein (e.g., the training protein 402) and a compound and comparing the predicted match score 412 with observed real-world data. For instance, the multi-domain tractability system 102 utilizes a compound protein interaction machine learning model 408 to generate the predicted match score 412. In other words, the multi-domain tractability system 102 verifies the predicted match score 412 by comparing observed binding data 410 for the protein (e.g., the training protein) with the predicted match score 412 (e.g., to adjust the accuracy of a model in generating the predicted match score).

As just mentioned, the multi-domain tractability system 102 compares the observed binding data 410 with the predicted match score 412. As used herein, the term “observed binding data” refers to experimental measurements or observations that indicate information about an interaction between a protein and a molecule (e.g., a target compound). Specifically, the observed binding data 410 includes data relating to molecular affinity (e.g., strength of an interaction between a protein and another target), kinetics (e.g., a rate of association and dissociation between a protein and another target), and interactions at a binding site of a protein (e.g., selectivity of a protein for a particular molecular).

Moreover, as shown, the multi-domain tractability system 102 generates the ground truth protein-model tractability measure 414 from the comparison between the predicted match score 412 and the observed binding data 410 for the training protein. Additionally, from comparing the predicted protein-model tractability score 406 and the ground truth protein-model tractability measure 414, the multi-domain tractability system 102 determines a measure of loss 416. For instance, the multi-domain tractability system 102 utilizes the measure of loss 416 to modify parameters of the protein tractability machine learning model 404. For example, the multi-domain tractability system 102 can utilize back-propagation (and/or gradient descent) to modify parameters of the protein tractability machine learning model 404 (e.g., to reduce the measure of loss).

As used herein, the term “measure of loss” refers to a numerical value that indicates a difference between a prediction and actual values. Specifically, the multi-domain tractability system 102 utilizes the measure of loss during training to reduce/minimize the loss (e.g., which in turn results in predictions closer to the actual values). For example, the measure of loss 416 can include mean squared error (MSE), cross-entropy loss, hinge loss (SVM loss), Huber loss, binary cross-entropy loss, categorical cross-entropy loss, and Kullback-Leibler Divergence (KL divergence).

In one or more embodiments, the multi-domain tractability system 102 can further train the protein tractability machine learning model 404 to generate tractability scores for non-human species such as animals (e.g., model organisms utilized in laboratory environments) or agricultural organisms. Specifically, the multi-domain tractability system 102 trains the protein tractability machine learning model 404 to generate tractability scores to determine the accuracy of multi-domain models.

In some embodiments, the multi-domain tractability system 102 trains the protein tractability machine learning model 404 on multiple different species. Specifically, the multi-domain tractability system 102 utilizes the compound protein interaction machine learning model 408 to generate predicted match scores for different species and compares the different predicted match scores with observed binding data for the training proteins of different species. As such, the multi-domain tractability system 102 generates ground truth protein-model tractability measures for the proteins of the different species (e.g., non-human/agricultural species) and compares a predicted protein-model tractability score for the protein domain to further determine a measure of loss. In doing so, the multi-domain tractability system 102 trains the protein tractability machine learning model 404 for the protein domain with respect to multiple different species.

In some embodiments, the multi-domain tractability system 102 trains the protein tractability machine learning model 404 on only human species but utilizes the protein tractability machine learning model 404 to generate protein tractability scores for other species. For instance, in some embodiments, a client device can indicate to the multi-domain tractability system 102 to generate a protein tractability score for a specific protein with regards to a specific non-human/agricultural species. To illustrate, for a protein present in both animals and humans (e.g., hemoglobin), the multi-domain tractability system 102 generates a protein tractability score for the compound protein interaction machine learning model 408 with regards to hemoglobin but in an animal species. In some instances, the multi-domain tractability system 102 can show a comparison between the protein tractability score of a human and an animal.

In some embodiments, the multi-domain tractability system 102 trains multiple different protein tractability machine learning models. Specifically, the multi-domain tractability system 102 utilizes the techniques described in FIG. 4 to train the protein tractability machine learning model 404, however the multi-domain tractability system 102 trains a single protein tractability machine learning model for humans, another protein tractability machine learning for a first non-human species (e.g., animal, or agricultural), another protein tractability machine learning model for a second non-human species, and so forth.

In some embodiments, the multi-domain tractability system 102 trains the protein tractability machine learning model 404 on human species but fine-tunes the protein tractability machine learning model 404 on non-human species. Specifically, the initial training iterations are performed with respect to human data (e.g., observed binding data for humans) and after training is completed, the multi-domain tractability system 102 further provides non-human examples to the protein tractability machine learning model 404. In such instances, the multi-domain tractability system 102 modifies parameters of the protein tractability machine learning model 404 based on the non-human examples.

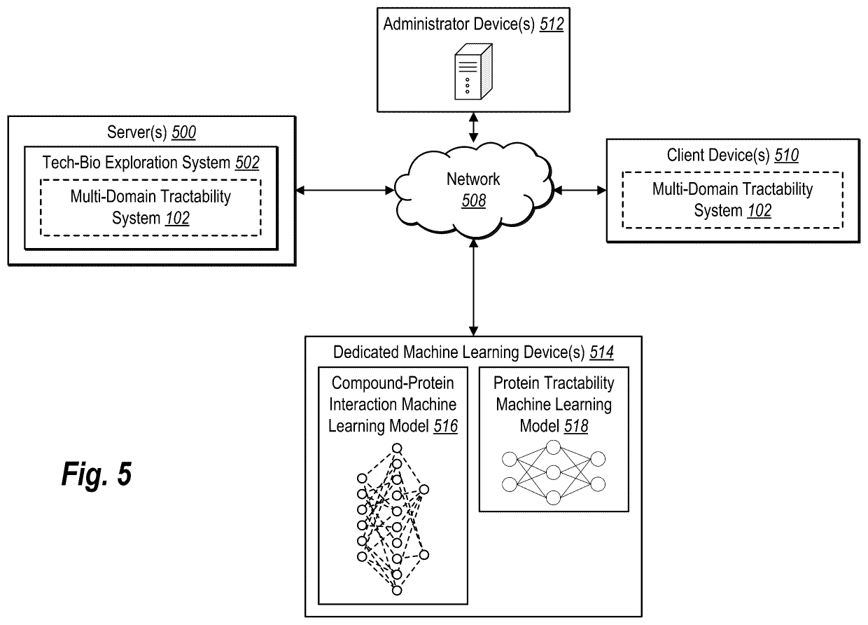

Additional detail regarding the multi-domain tractability system 102 environment will now be provided with reference to FIG. 5. In particular, FIG. 5 illustrates a schematic diagram of a system environment in which the multi-domain tractability system 102 can operate in accordance with one or more embodiments.

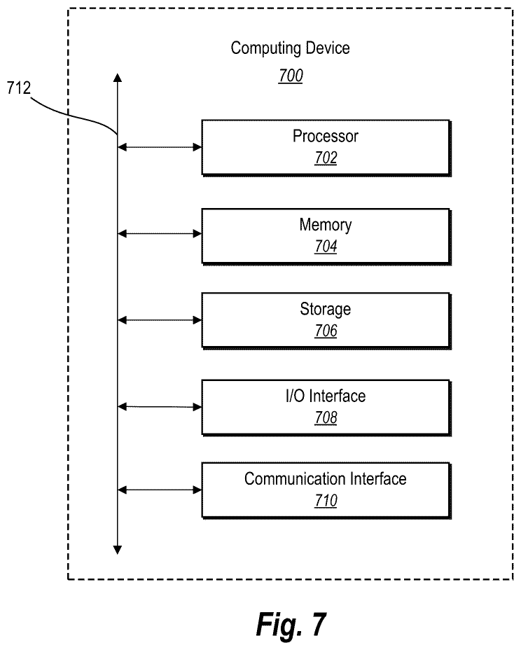

As shown in FIG. 5, the environment includes server(s) 500 (which includes a tech-bio exploration system 502 and the multi-domain tractability system 102), dedicated machine learning device(s) 514, a network 508, client device(s) 510 and administrator device(s) 512. As further illustrated in FIG. 5, the various computing devices within the environment can communicate via the network 508. Although FIG. 5 illustrates the multi-domain tractability system 102 being implemented by a particular component and/or device within the environment, the multi-domain tractability system 102 can be implemented, in whole or in part, by other computing devices and/or components in the environment (e.g., the additional device(s)). Additional description regarding the illustrated computing devices is provided with respect to FIG. 7 below.

As shown in FIG. 5, the server(s) 500 (e.g., one or more local servers operated by a particular entity) can include the tech-bio exploration system 502. In some embodiments, the tech-bio exploration system 502 can determine, store, generate, and/or display tech-bio information including maps of biology, experiments from various sources, and/or machine learning tech-bio predictions. For instance, the tech-bio exploration system 502 can analyze data signals corresponding to various treatments or interventions (e.g., compounds or biologics) and the corresponding relationships in genetics, proteomics, phenomics (i.e., cellular phenotypes), and invivomics (e.g., expressions or results within a living animal). Moreover, the tech-bio exploration system 502 provides an environment for operating, executing, and managing complex drug discovery pipelines.

For instance, the tech-bio exploration system 502 can generate and access experimental results corresponding to gene sequences, protein shapes/folding, protein/compound interactions, phenotypes resulting from various interventions or perturbations (e.g., gene knockout sequences or compound treatments), and/or invivo experimentation on various treatments in living animals. By analyzing these signals (e.g., utilizing various machine learning models), the tech-bio exploration system 502 can generate or determine a variety of predictions and inter-relationships for improving treatments/interventions.

To illustrate, the tech-bio exploration system 502 can generate maps of biology indicating biological inter-relationships or similarities between these various input signals to discover potential new treatments as part of the complex compound discovery process. For example, the tech-bio exploration system 502 can utilize machine learning and/or maps of biology to identify a similarity between a first gene associated with disease treatment and a second gene previously unassociated with the disease based on a similarity in resulting phenotypes from gene knockout experiments. The tech-bio exploration system 502 can then identify new treatments based on the gene similarity (e.g., by targeting compounds the impact the second gene). Similarly, the tech-bio exploration system 502 can analyze signals from a variety of sources (e.g., protein interactions, or invivo experiments) to predict efficacious treatments based on various levels of biological data.

The tech-bio exploration system 502 can generate GUIs comprising dynamic user interface elements to convey tech-bio information and receive user input for intelligently exploring tech-bio information. Indeed, as mentioned above, the tech-bio exploration system 502 can generate GUIs displaying different maps of biology that intuitively and efficiently express complex interactions between different biological systems for identifying improved treatment solutions. Furthermore, the tech-bio exploration system 502 can also electronically communicate tech-bio information between various computing devices.

As shown in FIG. 5, the tech-bio exploration system 502 can include a system that facilitates various models or algorithms for generating maps of biology (e.g., maps or visualizations illustrating similarities or relationships between genes, proteins, diseases, compounds, and/or treatments) and discovering new treatment options over one or more networks. For example, the tech-bio exploration system 502 collects, manages, and transmits data across a variety of different entities, accounts, and devices. In some cases, the tech-bio exploration system 502 is a network system that facilitates access to (and analysis of) tech-bio information within a centralized operating system. Indeed, the tech-bio exploration system 502 can link data from different network-based research institutions to generate and analyze maps of biology.

As shown in FIG. 5, the tech-bio exploration system 502 can include a system that comprises the multi-domain tractability system 102 that generates, stores, manages, transmits data pertaining to the tractability or confidence of a domain space for multi-domain models (e.g., and further generates bioactivity predictions). For example, in context of the above description for the tech-bio exploration system 502, in some embodiments the tech-bio exploration system 502 further utilizes the multi-domain tractability system 102 to enhance the coordination between various groups involved in the drug discovery process. For instance, the multi-domain tractability system 102 works in tandem with the tech-bio exploration system 502 to generate tractability scores, transmit the tractability scores to one or more devices, generate bioactivity predictions, and initiate one or more downstream model predictions or processes.

As also illustrated in FIG. 5, the environment includes the client device(s) 510. As mentioned above, the client device(s) 510 can be involved in the process of drug discovery. Thus, for example, the client device(s) 510 can coordinate/manage a first stage of generating a predicted match score (for a protein and a compound) and further generating a tractability score for the protein. Moreover, the client device(s) 510 can coordinate/manage a second stage such as generating a bioactivity prediction based on the tractability score. Further, the client device(s) 510 can coordinate/manage a third stage of utilizing the bioactivity prediction to generate one or more additional predictions or initiate one or more programs (IPG or ICG).

To illustrate, the client device(s) 510 can include computing devices that implement or manage a compound program generation stage of a compound discovery process. Similarly, the client device(s) 510 can include computing devices that implement or manage a compound lead generation stage and the client device(s) 510 can include computing devices that implement or manage a compound/dose selection stage. For example, the multi-domain tractability system 102 can receive one or more requests to utilize the dedicated machine learning device(s) 514 to generate one or more tractability scores. For instance, the multi-domain tractability system 102 can receive additional requests from the client device(s) 510 that include generating the bioactivity predictions.

In some embodiments, the environment also includes additional device(s). For example, the multi-domain tractability system 102 can utilize the additional device(s) to further operate and manage the completion of complex drug discovery pipelines. For instance, the additional device(s) include experimental device(s) and analytical device(s). Further, in some instances, the additional device(s) also include the computing devices discussed below in FIG. 7.

Furthermore, in one or more implementations, the client device(s) 510 include a client application. The client application can include instructions that (upon execution) cause the client device(s) 510 to perform various actions. For example, a user of a user account can interact with the client application on the client device(s) 510 to execute experiments or other multi-faceted processes and to further access tech-bio information, initiate a request for a protein-model tractability score, or a bioactivity prediction. For instance, in some embodiments the multi-domain tractability system 102 receives a request to generate a tractability score, and in response generates the score and returns the score to the client device(s) 510. In some instances, the transmittal of the tractability score to the client device(s) 510 causes the client device(s) 510 to execute an action (e.g., generate a downstream model prediction).

As shown, the environment can also include dedicated machine learning device(s) 514. For example, the dedicated machine learning device(s) 514 can include computing devices or virtual machines dedicated to training or implementing large-scale machine learning models. For example, the dedicated machine learning device(s) 514 can generate machine learning predictions and/or embeddings based on digital biological data (e.g., digital images of phenotypes resulting from different perturbations or compound-protein interactions from compound features). As shown, the dedicated machine learning device(s) 514 include a compound-protein interaction machine learning model 516 and a protein tractability machine learning model 518. Thus, the multi-domain tractability system 102 interacts with the dedicated machine learning device(s) 514 to generate the predicted match score and the protein-model tractability score.

The environment can also include experimental device(s). For example, the tech-bio exploration system 502 can interact with the experimental device(s) that include intelligent robotic devices and camera devices for generating and capturing digital images of cellular phenotypes resulting from different perturbations (e.g., genetic knockouts or compound treatments of stem cells). Similarly, the experimental device(s) can include camera devices and/or other sensors (e.g., heat or motion sensors) capturing real-time information from animals as part of invivo experimentation. The tech-bio exploration system 502 can also interact with a variety of other experimental device(s) such as devices for determining, generating, or extracting gene sequences or protein information. For example, the experimental device(s) may include computing devices linked to biosensorselectrophysiological platforms, x-ray crystallography machines, liquid chromatography mass spectrometry systems, nuclear magnetic resonance spectrometers, mass spectrometers. In some implementations, the multi-domain tractability system 102 generates the tractability scores and further determines to employ or utilize one or more experimental devices (e.g., to initiate one or more experiments based on the tractability scores).

As further shown in FIG. 5, the environment includes the network 508. As mentioned above, the network 508 can enable communication between components of the environment. In one or more embodiments, the network 508 may include a suitable network and may communicate using a various number of communication platforms and technologies suitable for transmitting data and/or communication signals, examples of which are described with reference to FIG. 7. Furthermore, although FIG. 5 illustrates computing devices communicating via the network 508, the various components of the environment can communicate and/or interact via other methods (e.g., communicate directly).

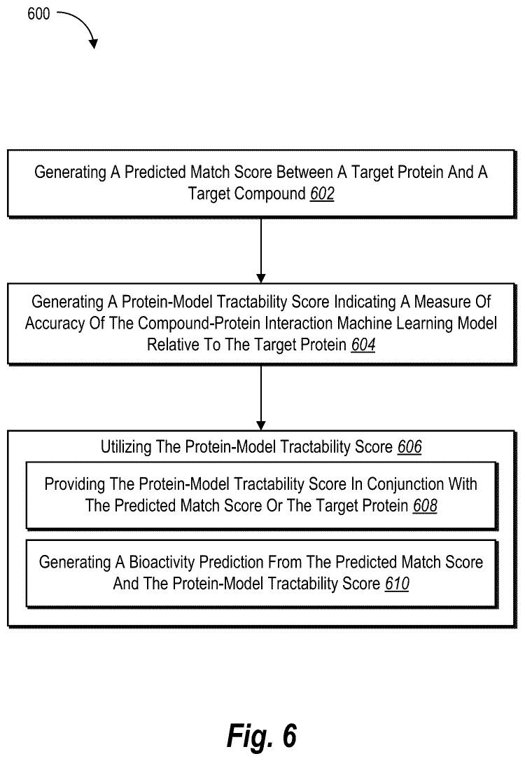

FIGS. 1-5, the corresponding text, and the examples provide a number of different systems, methods, and non-transitory computer readable media for generating a protein-model tractability score and utilizing the protein-model tractability score to provide to one or more computing devices or to generate a bioactivity prediction. In addition to the foregoing, embodiments can also be described in terms of flowcharts comprising acts for accomplishing a particular result. For example, FIG. 6 illustrates a flowchart of an example sequence of acts in accordance with one or more embodiments.

While FIG. 6 illustrates acts according to some embodiments, alternative embodiments may omit, add to, reorder, and/or modify any of the acts shown in FIG. 6. The acts of FIG. 6 can be performed as part of a method (e.g., a computer-implemented method). Alternatively, a non-transitory computer readable medium can comprise instructions, that when executed by one or more processors (e.g., at least one processor), cause a computing device to perform the acts of FIG. 6. In still further embodiments, a system can perform the acts of FIG. 6. Additionally, the acts described herein may be repeated or performed in parallel with one another or in parallel with different instances of the same or other similar acts.

FIG. 6 illustrates an example series of acts 600 for generating a protein-model tractability score in accordance with one or more embodiments. The series of acts 600 can include acts 602-610 of generating a predicted match score between a target protein and a target compound; generating a protein-model tractability score indicating a measure of accuracy of the compound-protein interaction machine learning model relative to the target protein, utilizing the protein-model tractability score; providing the protein-model tractability score in conjunction with the predicted match score or the target protein; or generating a bioactivity prediction from the predicted match score and the protein-model tractability score.

For example, in one or more embodiments, the acts 602-610 include generating, utilizing a compound-protein interaction machine learning model, a predicted match score between a target protein and a target compound; generating, utilizing a protein tractability machine learning model, a protein-model tractability score indicating a measure of accuracy of the compound-protein interaction machine learning model relative to the target protein; utilizing the protein-model tractability score by: providing, for display via a client device, the protein-model tractability score in conjunction with the predicted match score or the target protein; or generating, utilizing an additional prediction model, a bioactivity prediction from the predicted match score and the protein-model tractability score.

In one or more implementations, the series of acts 600 include generating a first vector representation from features of the target compound and a second vector representation from features of the target protein; and generating the predicted match score from the first vector representation and the second vector representation.

In addition, in one or more implementations, the series of acts 600 includes training the protein tractability machine learning model by: generating utilizing the protein tractability machine learning model, a predicted protein-model tractability score for the compound-protein interaction machine learning model and a training protein; comparing the predicted protein-model tractability score to a ground truth protein-model tractability measure to determine a measure of loss; and modifying parameters of the protein tractability machine learning model based on the measure of loss.

Further, in some implementations, the series of acts 600 includes generating the ground truth protein-model tractability measure by: generating, utilizing the compound-protein interaction machine learning model, predicted match scores for a protein and compounds; and comparing the predicted match score with observed binding data for the protein.

In one or more implementations, the series of acts 600 includes generating the bioactivity prediction from the predicted match score and the protein-model tractability score by comparing the protein-model tractability score to a protein tractability threshold. Moreover, in one or more implementations, the series of acts 600 includes generating the bioactivity prediction from the predicted match score and the protein-model tractability score by applying a first weight utilizing the protein-model tractability score.

In addition, in some implementations, the series of acts 600 includes generating at least one of: a program initiation rating, an ADMET prediction, or a compound exploration program performance prediction.

Further, in one or more implementations, the series of acts 600 includes generating, utilizing the protein tractability machine learning model, an additional protein-model tractability score indicating the measure of accuracy of the compound-protein interaction machine learning model relative to an additional target protein. In some implementations, the series of acts 600 includes utilizing the additional protein-model tractability score by: providing, for display via the client device, the additional protein-model tractability score; or generating, utilizing the additional prediction model, an additional bioactivity prediction from the additional protein-model tractability score.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 7 illustrates a block diagram of an example computing device 700 that may be configured to perform one or more of the processes described above. One will appreciate that one or more computing devices, such as the computing device 700 may represent the computing devices described above. In one or more embodiments, the computing device 700 may be a mobile device (e.g., a mobile telephone, a smartphone, a PDA, a tablet, a laptop, a camera, a tracker, a watch, a wearable device, etc.). In some embodiments, the computing device 700 may be a non-mobile device (e.g., a desktop computer or another type of client device). Further, the computing device 700 may be a server device that includes cloud-based processing and storage capabilities.

As shown in FIG. 7, the computing device 700 can include one or more processor(s) 702, memory 704, a storage device 706, input/output interfaces 708 (or “I/O interfaces 708”), and a communication interface 710, which may be communicatively coupled by way of a communication infrastructure (e.g., bus 712). While the computing device 700 is shown in FIG. 7, the components illustrated in FIG. 7 are not intended to be limiting. Additional or alternative components may be used in other embodiments. Furthermore, in certain embodiments, the computing device 700 includes fewer components than those shown in FIG. 7. Components of the computing device 700 shown in FIG. 7 will now be described in additional detail.

In particular embodiments, the processor(s) 702 includes hardware for executing instructions, such as those making up a computer program. As an example, and not by way of limitation, to execute instructions, the processor(s) 702 may retrieve (or fetch) the instructions from an internal register, an internal cache, memory 704, or a storage device 706 and decode and execute them.

The computing device 700 includes memory 704, which is coupled to the processor(s) 702. The memory 704 may be used for storing data, metadata, and programs for execution by the processor(s). The memory 704 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 704 may be internal or distributed memory.

The computing device 700 includes a storage device 706 includes storage for storing data or instructions. As an example, and not by way of limitation, the storage device 706 can include a non-transitory storage medium described above. The storage device 706 may include a hard disk drive (HDD), flash memory, a Universal Serial Bus (USB) drive or a combination these or other storage devices.

As shown, the computing device 700 includes one or more I/O interfaces 708, which are provided to allow a user to provide input to (such as user strokes), receive output from, and otherwise transfer data to and from the computing device 700. These I/O interfaces 708 may include a mouse, keypad or a keyboard, a touch screen, camera, optical scanner, network interface, modem, other known I/O devices or a combination of such I/O interfaces 708. The touch screen may be activated with a stylus or a finger.

The I/O interfaces 708 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 708 are configured to provide graphical data to a display for presentation to a user. The graphical data may be representative of one or more graphical user interfaces and/or any other graphical content as may serve a particular implementation.

The computing device 700 can further include a communication interface 710. The communication interface 710 can include hardware, software, or both. The communication interface 710 provides one or more interfaces for communication (such as, for example, packet-based communication) between the computing device and one or more other computing devices or one or more networks. As an example, and not by way of limitation, communication interface 710 may include a network interface controller (NIC) or network adapter for communicating with an Ethernet or other wire-based network or a wireless NIC (WNIC) or wireless adapter for communicating with a wireless network, such as a WI-FI. The computing device 700 can further include a bus 712. The bus 712 can include hardware, software, or both that connects components of computing device 700 to each other.

In one or more implementations, various computing devices can communicate over a computer network. This disclosure contemplates any suitable network. As an example, and not by way of limitation, one or more portions of a network may include an ad hoc network, an intranet, an extranet, a virtual private network (“VPN”), a local area network (“LAN”), a wireless LAN (“WLAN”), a wide area network (“WAN”), a wireless WAN (“WWAN”), a metropolitan area network (“MAN”), a portion of the Internet, a portion of the Public Switched Telephone Network (“PSTN”), a cellular telephone network, or a combination of two or more of these.

In particular embodiments, the computing device 700 can include a client device that includes a requester application or a web browser, such as MICROSOFT INTERNET EXPLORER, GOOGLE CHROME, or MOZILLA FIREFOX, and may have one or more add-ons, plug-ins, or other extensions, such as TOOLBAR or YAHOO TOOLBAR. A user at the client device may enter a Uniform Resource Locator (“URL”) or other address directing the web browser to a particular server (such as server), and the web browser may generate a Hyper Text Transfer Protocol (“HTTP”) request and communicate the HTTP request to server. The server may accept the HTTP request and communicate to the client device one or more Hyper Text Markup Language (“HTML”) files responsive to the HTTP request. The client device may render a webpage based on the HTML files from the server for presentation to the user. This disclosure contemplates any suitable webpage files. As an example, and not by way of limitation, webpages may render from HTML files, Extensible Hyper Text Markup Language (“XHTML”) files, or Extensible Markup Language (“XML”) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a webpage encompasses one or more corresponding webpage files (which a browser may use to render the webpage) and vice versa, where appropriate.

In particular embodiments, the tech-bio exploration system 502 may include a variety of servers, sub-systems, programs, modules, logs, and data stores. In particular embodiments, the tech-bio exploration system 502 may include one or more of the following: a web server, action logger, API-request server, transaction engine, cross-institution network interface manager, notification controller, action log, third-party-content-object-exposure log, inference module, authorization/privacy server, search module, user-interface module, user-profile (e.g., provider profile or requester profile) store, connection store, third-party content store, or location store. The tech-bio exploration system 502 may also include suitable components such as network interfaces, security mechanisms, load balancers, failover servers, management-and-network-operations consoles, other suitable components, or any suitable combination thereof. In particular embodiments, the tech-bio exploration system 502 may include one or more user-profile stores for storing user profiles and/or account information for credit accounts, secured accounts, secondary accounts, and other affiliated financial networking system accounts. A user profile may include, for example, biographic information, demographic information, financial information, behavioral information, social information, or other types of descriptive information, such as interests, affinities, or location.

The web server may include a mail server or other messaging functionality for receiving and routing messages between the tech-bio exploration system 502 and one or more client devices. An action logger may be used to receive communications from a web server about a user's actions on or off the tech-bio exploration system 502. In conjunction with the action log, a third-party-content-object log may be maintained of user exposures to third-party-content objects. A notification controller may provide information regarding content objects to a client device. Information may be pushed to a client device as notifications, or information may be pulled from a client device responsive to a request received from the client device. Authorization servers may be used to enforce one or more privacy settings of the users of the tech-bio exploration system 502. A privacy setting of a user determines how particular information associated with a user can be shared. The authorization server may allow users to opt in to or opt out of having their actions logged by the tech-bio exploration system 502 or shared with other systems, such as, for example, by setting appropriate privacy settings. Third-party-content-object stores may be used to store content objects received from third parties. Location stores may be used for storing location information received from a client device associated with users.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
generating, utilizing a compound-protein interaction machine learning model, a predicted match score between a target protein and a target compound;
generating, utilizing a protein tractability machine learning model, a protein-model tractability score indicating a measure of accuracy of the compound-protein interaction machine learning model relative to the target protein; and
utilizing the protein-model tractability score by:
providing, for display via a client device, the protein-model tractability score in conjunction with the predicted match score or the target protein; or
generating, utilizing an additional prediction model, a bioactivity prediction from the predicted match score and the protein-model tractability score.

2. The computer-implemented method of claim 1, wherein generating the predicted match score between the target protein and the target compound further comprises:
generating a first vector representation from features of the target compound and a second vector representation from features of the target protein; and
generating the predicted match score from the first vector representation and the second vector representation.

3. The computer-implemented method of claim 1, further comprising training the protein tractability machine learning model by:
generating utilizing the protein tractability machine learning model, a predicted protein-model tractability score for the compound-protein interaction machine learning model and a training protein;
comparing the predicted protein-model tractability score to a ground truth protein-model tractability measure to determine a measure of loss; and
modifying parameters of the protein tractability machine learning model based on the measure of loss.

4. The computer-implemented method of claim 3, further comprising generating the ground truth protein-model tractability measure by:
generating, utilizing the compound-protein interaction machine learning model, predicted match scores for a protein and compounds; and
comparing the predicted match score with observed binding data for the protein.

5. The computer-implemented method of claim 1, further comprising generating the bioactivity prediction from the predicted match score and the protein-model tractability score by comparing the protein-model tractability score to a protein tractability threshold.

6. The computer-implemented method of claim 1, further comprising generating the bioactivity prediction from the predicted match score and the protein-model tractability score by applying a first weight utilizing the protein-model tractability score.

7. The computer-implemented method of claim 1, wherein generating the bioactivity prediction from the predicted match score and the protein-model tractability score comprises generating at least one of: a program initiation rating, an ADMET prediction, or a compound exploration program performance prediction.

8. The computer-implemented method of claim 1, further comprising generating, utilizing the protein tractability machine learning model, an additional protein-model tractability score indicating the measure of accuracy of the compound-protein interaction machine learning model relative to an additional target protein.

9. The computer-implemented method of claim 8, further comprising utilizing the additional protein-model tractability score by:
providing, for display via the client device, the additional protein-model tractability score; or
generating, utilizing the additional prediction model, an additional bioactivity prediction from the additional protein-model tractability score.

10. A system comprising:
at least one processor; and
at least one non-transitory computer-readable storage medium storing instructions that, when executed by the at least one processor, cause the system to:
generate, utilizing a compound-protein interaction machine learning model, a predicted match score between a target protein and a target compound;
generate, utilizing a protein tractability machine learning model, a protein-model tractability score indicating a measure of accuracy of the compound-protein interaction machine learning model relative to the target protein; and
utilize the protein-model tractability score by:
providing, for display via a client device, the protein-model tractability score in conjunction with the predicted match score or the target protein; or
generating, utilizing an additional prediction model, a bioactivity prediction from the predicted match score and the protein-model tractability score.

11. The system of claim 10, further comprising instructions that, when executed by the at least one processor, cause the system to generate the predicted match score between the target protein and the target compound by:
generating a first vector representation from features of the target compound and a second vector representation from features of the target protein; and
generating the predicted match score from the first vector representation and the second vector representation.

12. The system of claim 10, further comprising instructions that, when executed by the at least one processor, cause the system to train the protein tractability machine learning model by:
generating utilizing the protein tractability machine learning model, a predicted protein-model tractability score for the compound-protein interaction machine learning model and a training protein;
comparing the predicted protein-model tractability score to a ground truth protein-model tractability measure to determine a measure of loss; and
modifying parameters of the protein tractability machine learning model based on the measure of loss.

13. The system of claim 10, further comprising instructions that, when executed by the at least one processor, cause the system to generate the bioactivity prediction from the predicted match score and the protein-model tractability score by comparing the protein-model tractability score to a protein tractability threshold.

14. The system of claim 10, further comprising instructions that, when executed by the at least one processor, cause the system to generate the bioactivity prediction from the predicted match score and the protein-model tractability score by applying a first weight utilizing the protein-model tractability score.

15. The system of claim 10, further comprising instructions that, when executed by the at least one processor, cause the system to generate the bioactivity prediction from the predicted match score and the protein-model tractability score, which comprises generating at least one of: a program initiation rating, an ADMET prediction, or a compound exploration program performance prediction.

16. A non-transitory computer-readable medium storing instructions that, when executed by at least one processor, cause a computing device to:
generate, utilizing a compound-protein interaction machine learning model, a predicted match score between a target protein and a target compound;
generate, utilizing a protein tractability machine learning model, a protein-model tractability score indicating a measure of accuracy of the compound-protein interaction machine learning model relative to the target protein; and
utilize the protein-model tractability score by:
providing, for display via a client device, the protein-model tractability score in conjunction with the predicted match score or the target protein; or
generating, utilizing an additional prediction model, a bioactivity prediction from the predicted match score and the protein-model tractability score.

17. The non-transitory computer-readable medium of claim 16, further comprising instructions that, when executed by the at least one processor, cause the computing device to:
generate a first vector representation from features of the target compound and a second vector representation from features of the target protein; and
generate the predicted match score from the first vector representation and the second vector representation.

18. The non-transitory computer-readable medium of claim 16, further comprising instructions that, when executed by the at least one processor, cause the computing device to:
train the protein tractability machine learning model by:
generating utilizing the protein tractability machine learning model, a predicted protein-model tractability score for the compound-protein interaction machine learning model and a training protein;
comparing the predicted protein-model tractability score to a ground truth protein-model tractability measure to determine a measure of loss; and
modifying parameters of the protein tractability machine learning model based on the measure of loss.

19. The non-transitory computer-readable medium of claim 16, further comprising instructions that, when executed by the at least one processor, cause the computing device to generate the bioactivity prediction from the predicted match score and the protein-model tractability score by applying a first weight utilizing the protein-model tractability score.

20. The non-transitory computer-readable medium of claim 16, further comprising instructions that, when executed by the at least one processor, cause the computing device to generate the bioactivity prediction from the predicted match score and the protein-model tractability score by generating at least one of: a program initiation rating, an ADMET prediction, or a compound exploration program performance prediction.
